(19) United States
(12) Patent Application Publication
(10) Pub. No.: US 2006/0161231 A1
(43) Pub. Date: Jul. 20, 2006

(54) Non-occlusion convective blanket

(75) Inventors: Karl Hans Cazzini, Orchard Park, NY (US); Joel T. Jusiak, Holland, NY (US)

Correspondence Address: Kevin D. McCarthy;Roach Brown McCarthy & Gruber, P.C., 1620 Liberty Building, 420 Main Street, Buffalo, NY, 14202, US

(73) Assignee: Gaymar Industries, Inc.

(21) Appl. No.: 11/037,066

(22) Filed: Jan. 17, 2005

(51) Int. Cl. A61F 7/00

(52) U.S. Cl. 607/104

(57) ABSTRACT

A thermal treatment apparatus for underlying and/or covering and bathing a patient in a thermally-controlled inflating fluid is disclosed. The apparatus has an inflatable chamber, fluid inlet, and a plurality of interspaced exhaust ports. The inflatable chamber is designed to receive or overlie at least a portion of a patient and is defined by at least one upper surface material and a lower surface material. The fluid inlet is disposed in the inflatable chamber for receiving a thermally-controlled inflating fluid into the inflatable chamber. Where the inflating fluid is a gas, including and not limited to air, the plurality of interspaced exhaust ports are formed on the inflatable chamber for exhausting the inflating fluid from the inflatable chamber toward the portion of the patient position contacting the inflatable chamber. The exhaust ports can be positioned along the seal of the upper and lower materials and within an interior aperture positioned in the inflatable chamber. Filler can also be positioned within the inflatable chamber to provide support to decrease the chance of occlusion in the inflatable chamber.

### FIELD OF THE INVENTION

The present invention is directed to a convective blanket.

### BACKGROUND OF THE PRESENT INVENTION

Inflatable thermal blankets are used to communicate a conditioned gas, such as heated or cooled air, to a patient and are known in the art. Such thermal blankets typically have an inflatable portion provided with an inlet port for placing the inflatable portion in fluid communication with a source of pressurized, conditioned gas such that the inflatable portion can be selectively inflated. The inflatable portion generally has an inner surface which is gas pervious, or which is otherwise adapted to communicate the conditioned gas used to inflate the blanket to the user. Such thermal blankets are often used to treat conditions such as hypothermia, or used to reduce the body temperature of a user in circumstances where the body temperature is inappropriately high. For example, where a patient is being treated for hypothermia, at least a portion of the patient's body is covered with and/or overlies the thermal blanket, and warm air is pumped into the inflatable portion. The warm air used to inflate the inflatable portion is thereafter communicated through the inner surface of the inflatable portion so as to bath the body portion covered and/or overlaid by the blanket in warm air.

Examples of such thermal blankets are disclosed in Augustine Medical, Inc. v. Gaymar Indus., Inc., 181 F.3d 1291, 50 USPQ2d 1900 (Fed. Cir. 1999). In that decision, the Federal Circuit stated “[Gaymar's convective] blankets feature an inflatable quilt-like structure. The [Gaymar] blankets attach two sheets of the same amount of flexible, lightweight material around their periphery and at various spots along their surfaces. In operation, heated air flows onto a patient's body from holes in the undersurface of the accused blankets, but the blankets do not form a self-supporting or Quonset hut-like structure. Instead, the accused blankets lie flat when inflated on a flat surface and rest substantially on a patient when in use. . . . Gaymar began selling forced-air blankets in March 1992.”

Gaymar has recognized that its convective air blankets have been positioned underneath patients, in particular infants, to obtain similar results. Other entities have attempted to capitalize on this use of the convective blankets. They have modified the blanket for just an infant, as illustrated in the Bair Hugger pediatric underbody blanket, model 555. That blanket has two drapes to form a tent of warm air that surround the patient. There are hose ports at either end of the blanket to provide options for hose placement. There is an adhesive on the bottom of that blanket and wings that are designed to tuck under the operating room table for stability. That blanket also has fluid outlets that minimize the pooling of fluids on the blanket's surface. That blanket, however, has problems because the warm air is unable, sometimes, to exhaust through all the holes due to occlusion caused by the patient. That means the warm air is sometimes directed to other holes that may overheat the patient in certain areas and under heat the patient in other areas. A cause of that problem may be the patient's weight on the blanket is greater than the air pressure penetrating into that area of the blanket. Another cause of that problem may be the patient occludes at least some of the apertures.

In U.S. Pat. No. 6,156,058; Kappel et al. disclose another variation of a pediatric convective blanket that is positioned above and/or below the patient. The Kappel et al. blanket is similar to Gaymar's original convective blanket design except it is smaller and it has at least two inlets. Kappel et al. recognizes that their blanket has problems. In particular, Kappel et al. wrote, “When placing blankets under patients, it is often the case that the weight of the patient will cause air flow to be partially or completely restricted through certain portions of the blanket. Therefore, it is desirable to include means of providing for greater and more consistent air flow through the blanket in order to supply the same amount of warming therapy to the patient.” That problem is the same problem that we disclosed for the model 555 blanket.

According to Kappel et al. that problem was solved, “By providing multiple inlet ports and exit vents, the circulating warm air has more pathways to move through the blanket and thus provide a greater and more consistent airflow through the blanket.” Notice that the model 555 and the Kappel et al. design utilize multiple inlet ports.

That solution, however, does not address other problems that are associated with patients, especially the elderly and the real young. Both sets of patients have potential incontinence issues. If a patient had incontinence issues on the blankets of Kappel et al. and/or model 555, the patient could theoretically be contacted with that incontinence issue. That is an unacceptable outcome.

Other devices for warming or cooling patients, and/or for communicating conditioned air to a patient, are disclosed in U.S. Pat. Nos. 1,777,982; 2,093,834; 3,653,083; 4,347,633; 4,472,847; 4,572,188; 4,660,388; 4,777,802; 5,106,373; 5,165,400; 5,300,101; 5,300,102; 5,336,250; 5,350,417; 5,405,371; 5,545,194; 5,674,269; 5,860,292; 6,102,936; 6,210,428 B1; 6,228,107 B1, and 6,511,501.

To address both or one of the above-identified problems, applicants have conceived and reduced to practice the disclosed invention.

### BRIEF DESCRIPTION OF THE FIGURES

These and other important objectives and advantages will become evident when the detailed description of the invention is read with reference to the below-summarized drawings, in which:

FIG. 1 is an overview of one embodiment of the present invention.

FIG. 2 is a cross-sectional view of FIG. 1 taken along the lines 2-2 and illustrates the prior art.

FIGS. 3a and 3b illustrate embodiments of the present invention of FIG. 1 taken along the lines 2-2.

FIGS. 4a and 4b are embodiments of the present invention.

FIG. 5 is an alternative embodiment of the present invention taken from FIG. 1 at box 5.

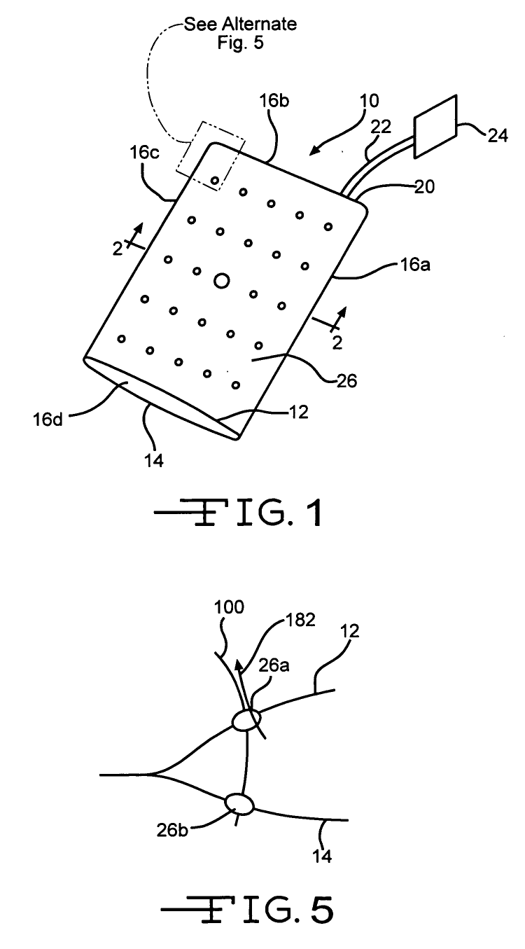

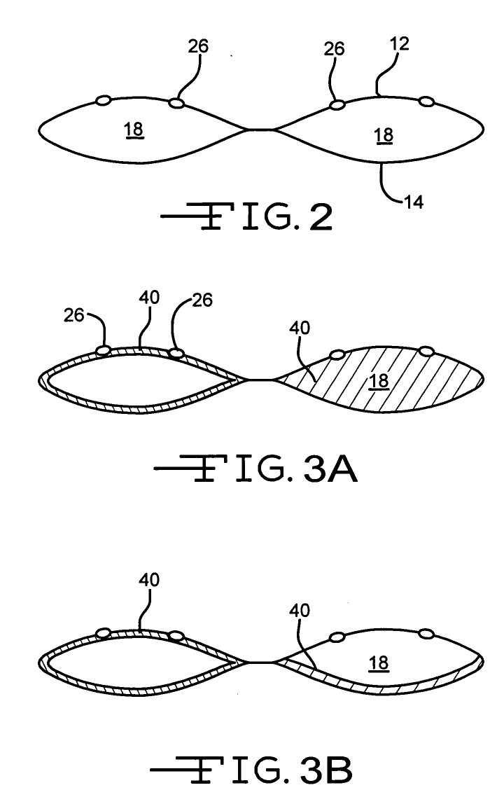

### SUMMARY OF THE INVENTION

A thermal treatment apparatus for underlying and/or covering and bathing a patient in a thermally-controlled inflating fluid is disclosed. The apparatus has an inflatable chamber, fluid inlet, and a plurality of interspaced exhaust ports. The inflatable chamber is designed to receive or overlie at least a portion of a patient and is defined by at least one upper surface material and a lower surface material. The fluid inlet is disposed in the inflatable chamber for receiving a thermally-controlled inflating fluid into the inflatable chamber. Where the inflating fluid is a gas, including and not limited to air, the plurality of interspaced exhaust ports are formed on the inflatable chamber for exhausting the inflating fluid from the inflatable chamber toward the portion of the patient position contacting the inflatable chamber. The exhaust ports can be positioned along the seal of the upper and lower materials and within an interior aperture positioned in the inflatable chamber. Filler can also be positioned within the inflatable chamber to provide support to decrease the chance of occlusion in the inflatable chamber.

### DETAILED DESCRIPTION OF THE INVENTION

The present invention is directed to a convective blanket 10. Each convective blanket 10 has similar characteristics. Those characteristics are an upper surface 12 of a cushion, a lower surface 14 of a cushion, an inflatable chamber 18, and an inlet 20.

The upper surface 12 and the lower surface 14 are sealed together on at least three sides 16a, 16b, 16c, and possibly a fourth side 16d. The seal can be obtained by sonic welding, heat welding, threaded together, adhesives, or any conventional method to join at least two materials together. In addition, the two surfaces 12, 14 can be joined together at numerous other locations throughout the device 10. These other seals are referred to as welded portions 15. These welded portions can come in numerous sizes (normally relatively small) and shapes (circles, finger-like projections, partial circles). These welded portions are designed to facilitate the distribution and circulation of the fluid throughout the inflatable chamber. The welded portions can also have slits therein. By having slits, the cushion can be altered into various designs to have one portion of the cushion on and/or over a portion of the patient and other portions not over and/or on the patient or variations thereof.

The materials used for the upper and lower surfaces may be formed of any suitable material capable of being sealed together at selected positions and having sufficient strength to allow inflation and adequate air distribution within the inflated air chamber. The materials used for the upper and lower surfaces include and are not limited to polymeric materials, metallic, natural fibers, and combinations thereof. The material is any material that can be used in association with a patient. A preferred material for the upper surface is spunbound non-woven polypropylene with 1 (one) mil of polyethylene coating on the interior surface. That means the preferred material for the upper surface is an air impermeable material. The bottom surface is also normally an air impermeable material but both materials could be, but not normally, air permeable materials. Air permeable materials are not equivalent to air impermeable materials for many reasons. One of those reasons is that air permeable materials allow the air to exhaust all over the place. Such erratic distribution from air permeable materials is undesirable that is why air impermeable material with positioned outlets, discussed below, that direct the fluid to the desired location of the patient is superior to air permeable materials. The upper surface and the lower surface can be the same material, or alternatively a plurality of the same and/or different materials.

In addition, the blankets may be formed of various laminated layers of the above materials. For example, each upper and/or lower surface of the blankets could be a single, two-, or multi-ply layer(s) of material.

The interior inflatable chamber 18 receives a fluid. The fluid is normally a gas, and preferably air, at a desired temperature—ambient, warm, hot, cool, or cold. The fluid enters the at least one inlet 20 of the convective blanket 10 through a conduit 22 from a source 24. The source 24 can be any device that has and/or generates a pressurized fluid that can inflate and circulate through the inflatable chamber 18.

The fluid circulates within the interior inflatable chamber 18 and exhausts through at least one outlet 26 positioned on either or both of the upper and/or lower surfaces 12, 14. Preferably there are various outlets 26 interspaced on at least one surface 12, 14 (normally the upper surface 12) of the convective blanket 10.

In a preferred embodiment, the inlet 22 directs the fluid to a patient, not shown. The convective blanket 10 can be positioned above or below the patient. All of the above information is admittedly prior art and Gaymar has made versions of this product since 1992 as a blanket, a mattress, and a cushion. Obvious variations of these devices include inflatable collars, and devices positioned around the perimeter, or at least partially around the perimeter of a patient to apply a fluid to the patient for the same reasons as disclosed by Gaymar since 1992. As such, all these devices are collectively referred to as a “convective blanket” for this application.

In another embodiment of the present invention, the convective blankets may be treated with a fire retardant material. In particular, the blankets may be treated by a spraying, coating, or other appropriate technique, with the fire retardant material. Such treatment produces advantageous blankets which are both nonflammable and laser resistant. Such characteristics may be desirable for hospital settings. The fire retardant spray may be any suitable spray which will render the blanket nonflammable and laser resistant. Such a spray may be used regardless of the material from which the blanket is made. One material which has been found to be useful as a fire retardant spray for convective air warming blankets is available from Project Fire Safety, Inc. and is identified as product number MG 702. The treatment of blankets to render them nonflammable and laser resistant is applicable to pediatric and adult sized blankets, as well as any other size that may be desired.

It should be noted that while particular blankets described above have been identified for use primarily in the operating room or for use primarily outside the operating room that it will be evident to one skilled in the art that any of the blankets according to the present invention could be used in areas other than the primarily indicated area. For example, a blanket for use primarily in the operating room could also be used outside the operating room, such as in the PACU, ICU or regular hospital room, and vice versa. Further, any of the blankets described herein could also be used in a nursing home, patient's home or any place where hypothermia is a problem.

Occlusion of the interior inflatable chamber is a problem to be avoided. If occlusion occurs, the patient will not receive the desired fluid application. Such results could be deleterious to the patient.

To avoid that problem, applicants insert a filler material 40, as shown in FIG. 3, into the interior inflatable chamber 18. The filler can encompass the entire interior chamber as illustrated in FIG. 3a, or alternatively on at least one of the interior upper and/or lower surfaces (which includes both surfaces) as illustrated in FIG. 3b. The filler is any material that (a) decreases the occlusion of the fluid from circulating throughout the interior inflatable chamber and (b) allows the fluid to circulate within at least a part of the inflatable chamber to exhaust through the outlets 26 even with a patient thereon. Examples of the filler can include and not be limited to polymeric materials, natural fibers and synthetic blends, and mixtures thereof. Specific examples include and are not limited to cotton, structural honeycomb materials from Tytex of Woonsocket, R.I.; wool, silk, rayon, polypropylene, cotton and polyester blends, polyester and cellulose blends, rayon and polyester blends, non-woven wood pulp compositions, laminated plastic and wood pulp materials, and combinations thereof. Those materials are positioned within the interior of the inflatable chamber.

The filler allows the fluid to circulate throughout the interior chamber 18. With the filler there is a decreased chance of occlusion occurring within the interior chamber even with a patient lying on the cushion 10. This is because the filler provides some support that allows the fluid to circulate through the interior chamber 18 when the patient (and/or object) is almost occluding the interior chamber.

The filler material 40 can also be impregnated throughout with or just be an absorbent material. An example of an absorbent material is sodium polyacrylate or variations thereof. The invention described herein can utilize these unique characteristics of polyacrylates for capturing incontinent liquids. These characteristics will prevent escape of such incontinent fluids from annoying and/or disturbing the patient and provide safety measures because of the manner the fluid is absorbed, adsorbed or bound. Super absorbents such as sodium polyacrylate will not only absorb many times its own weight of liquid but they also form a gel that binds the liquid to itself without a chemical reaction. Further, the resulting gel is elastic and is many times the volume of the polyacrylate and liquid themselves. This provides an expansion or swelling that stabilizes and immobilizes any escaping liquid from the patient as well as stabilizing the position of the patient on the cushion.

In addition, the provision of the cushion with a super absorbent material can determine the amount of the incontinent fluid by measuring the cushion in relation to a pre-used weight of the cushion (that value may be measured by the manufacturer and/or the applier (or some entity associated with the applier) of the blanket to the patient) prior to the use of the blanket with the patient. This weight measure system, which uses a conventional balance or other weight measuring device, can be beneficial to patients who must or should know the quantity of fluid that is dispersed.

The inlet is defined in the upper surface 12, the lower surface 14, at the seal 16, or combinations thereof. The inlet receives the conduit 22 that transports the fluid, preferably at least pressurized, from a source to the interior chamber.

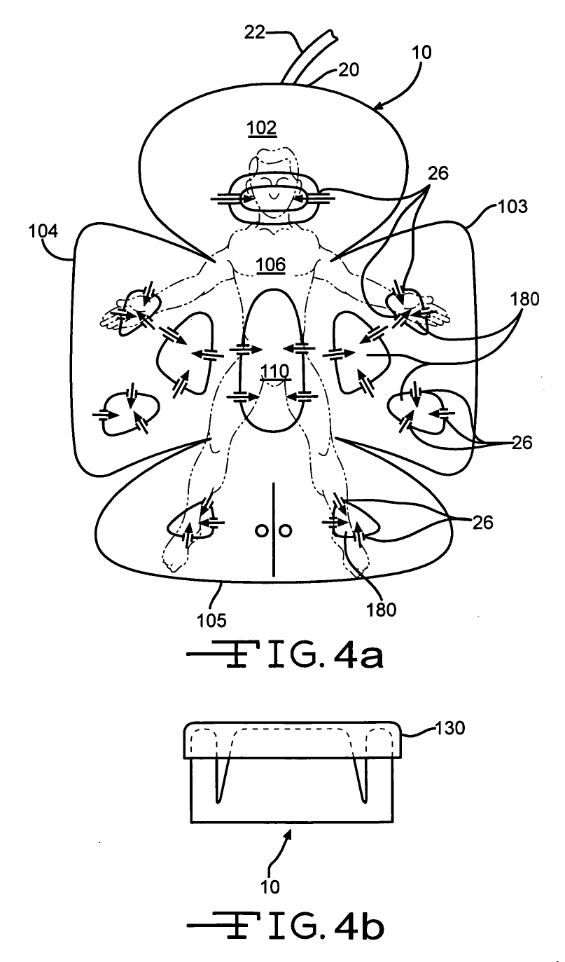

The cushion 10 can have any possible design. In one preferred embodiment, the cushion design is shaped like a cross having a top section 102, a left section 103, a right section 104, a bottom section 105 and a center section 106, as illustrated in FIG. 4a. The design embodiment of FIG. 4a is a preferred embodiment because it is shaped to conform into a cushion, a wrap-around blanket, and/or an incubator design, as shown in FIG. 4b, with the addition of a sheet 130 over the incubator design. The sheet should contain the thermal energy, and ensure the patient is properly wrapped in the desired thermal environment.

Alternative design embodiments can also provide an incubator design. Those designs can be a rectangular (which includes a square) shape, a circular shape, a triangular shape, a polygonal shape, and any three dimensional shape based on the identified two dimensional shapes or combinations thereof. Those various designs can have two or more slitted welded portions to allow these designs to form into an incubator. The cross design has just been found by the applicants to be a preferred design shape but by no means the exclusive shape that can be used.

In the center section 106 is at least an aperture 110. The aperture 110 is designed to at least receive at least one of the patient's incontinent dispersal units. That way the incontinent material (represented as arrow 180) from the dispersal unit can (a) miss the cushion 10, (b) be captured by the filler material 40 within the cushion 10 if the material enters and stays within the cushion, and/or (c) pass through the cushion if the material goes through two corresponding outlets 26a, 26b as illustrated in FIG. 5 and allows the thermal fluid (represented by arrow 182) to pass through to the patient.

The center aperture 110, and various other apertures that may be positioned about the cushion, has a plurality of fluid outlets 26. These outlets can be positioned along the seal that joins the upper surface and the lower surface of the cushion that forms the interior chamber as illustrated in FIG. 4a. These outlets may differ from other outlets. The difference can be that the outlet along the seal can be a semi-circle design. Why a semi-circle design? One reason is that as the inflatable chamber expands, the semi-shape (like a circle, rectangle, or polygon) design on the upper and lower surfaces becomes a complete shape. As a complete circle along the seam, there is a decreased chance the outlet will be occluded. That semi-circle design also concentrates the fluid blowing on to the patient, instead of dispersing the desired fluid to undesired locations.

Alternatively, the outlet can be a complete circle to allow the fluid to be directed toward the patient.

The blankets according to the present invention have been described as being useful in the prevention and treatment of hypothermia. It will be evident to one skilled in the art that a source of pressurized cooled air or room temperature air could also be provided to the blankets according to the present invention to control body temperature of the patient under conditions of hyperthermia.

The inflatable body 10 may be arranged around a patient in a variety of configurations to form a thermal care space. For example, the inflatable body 10 could be formed in a generally U-shaped configuration.

The fluid source can be from Gaymar's Thermacare device and/or Medi-Therm device. Both of these devices can control the temperature and the pressure, independently and/or collectively.

Preferably, the present invention is designed for an infant and/or pediatric patient but it can be used for older individuals, including adults.

Many modifications and variations of our invention will be evident to those skilled in the art. For example, thermal coverings for additional selected patient areas could be implemented depending on the location of the care site and the need for thermally maintaining other areas. It is understood that such variations may deviate from specific teachings of this description without departing from the essence of the invention, which is expressed in the following claims.

We claim:

1. A thermal treatment apparatus for covering and bathing a patient in a thermally-controlled inflating fluid, comprising:
an inflatable chamber designed to receive or overlie at least a portion of a patient, and defined by at least one upper surface material and a lower surface material;
a fluid inlet disposed in the inflatable chamber for receiving a thermally-controlled inflating fluid into the inflatable chamber;
a plurality of interspaced exhaust ports formed on the inflatable chamber for exhausting the inflating fluid from the inflatable chamber toward the portion of the patient position contacting the inflatable chamber; and
a filler within the inflatable chamber to provide support to decrease the chance of occlusion in the inflatable chamber.

2. The thermal treatment apparatus of claim 1 wherein the filler is selected from the group consisting of polymeric materials, natural fibers and synthetic blends, and mixtures thereof.

3. The thermal treatment apparatus of claim 1 wherein the filler is distributed throughout the interior chamber.

4. The thermal treatment apparatus of claim 1 wherein the filler is positioned on at least one of the interior surfaces of the interior chamber.

5. The thermal treatment apparatus of claim 1 wherein the filler comprises an absorbent material.

6. The thermal treatment apparatus of claim 1 wherein the fluid is a gas.

7. The thermal treatment apparatus of claim 6 wherein the gas is air.

8. The thermal treatment apparatus of claim 1 wherein the fluid is a liquid.

9. The thermal treatment apparatus of claim 1 wherein the fluid has a predetermined temperature.

10. The thermal treatment apparatus of claim 1 wherein the fluid has a predetermined pressure.

11. The thermal treatment apparatus of claim 1 further comprising corresponding outlets on the upper and lower surface materials.

12. The thermal treatment apparatus of claim 1 wherein the upper surface material and the lower surface material are sealed together at the periphery of the materials.

13. The thermal treatment apparatus of claim 12 wherein the upper surface material and lower surface material are sealed together at places other than the periphery.

14. The thermal treatment apparatus of claim 13 wherein there is at least one slit or aperture positioned where the upper surface material and lower surface material are sealed together at places other than the periphery.

15. The thermal treatment apparatus of claim 14 wherein the at least one slit or aperture is positioned to receive at least one of a patient's incontinent dispersal units.

16. The thermal treatment apparatus of claim 14 wherein the interspaced exhaust ports are positioned along the seal of the at least one slit or aperture.

17. The thermal treatment apparatus of claim 16 wherein the interspaced exhaust ports are semi-shape in design.

18. The thermal treatment apparatus of claim 16 wherein the interspaced exhaust ports are shaped in any conventional design.

19. The thermal treatment apparatus of claim 1 wherein the apparatus is capable of being shaped to form a partial incubator device around the patient.

20. The thermal treatment apparatus of claim 19 further comprising an incubator sheet that encloses the apparatus that is shaped into a partial incubator device to form an incubator unit.

21. The thermal treatment apparatus of claim 1 wherein the apparatus is designed for infants and pediatric uses.

22. The thermal treatment apparatus of claim 1 wherein the apparatus is designed for adults.

23. A thermal treatment apparatus for covering and bathing a patient in a thermally-controlled inflating fluid, comprising:
an inflatable chamber designed to receive or overlie at least a portion of a patient, and defined by at least one upper surface material and a lower surface material that are sealed together (a) at least along the periphery and (b) to form at least one interior aperture;
a fluid inlet disposed in the inflatable chamber for receiving a thermally-controlled inflating fluid into the inflatable chamber;
a plurality of interspaced exhaust ports formed on the seal that forms the at least one interior aperture.

24. The thermal treatment apparatus of claim 23 further comprising a filler within the inflatable chamber to provide support to decrease the chance of occlusion in the inflatable chamber.

25. The thermal treatment apparatus of claim 24 wherein the filler is selected from the group consisting of polymeric materials, natural fibers and synthetic blends, and mixtures thereof.

26. The thermal treatment apparatus of claim 24 wherein the filler is distributed throughout the interior chamber.

27. The thermal treatment apparatus of claim 24 wherein the filler is positioned on at least one of the interior surfaces of the interior chamber.

28. The thermal treatment apparatus of claim 24 wherein the filler comprises an absorbent material.

29. The thermal treatment apparatus of claim 23 wherein the fluid is a gas.

30. The thermal treatment apparatus of claim 29 wherein the gas is air.

31. The thermal treatment apparatus of claim 23 wherein the fluid is a liquid.

32. The thermal treatment apparatus of claim 23 wherein the fluid has a predetermined temperature.

33. The thermal treatment apparatus of claim 23 wherein the fluid has a predetermined pressure.

34. The thermal treatment apparatus of claim 23 further comprising corresponding outlets on the upper and lower surface materials.
